(19) United States
(12) Patent Application Publication
(10) Pub. No.: US 2012/0128845 A1
(43) Pub. Date: May 24, 2012

(54) Ice, toxicity, thermal-stress and cold-fracture management during cryopreserving encapsulation of specimens using controlled ice nucleation

(76) Inventor: Carol Ann Tosaya, Los Altos Hills, CA (US)

(21) Appl. No.: 12/927,757

(22) Filed: Nov. 22, 2010

(51) Int. Cl. A01N 1/02; C12N 5/04; A23L 3/375; C12M 1/00; C12N 5/07; B82Y 5/00

(52) U.S. Cl. 426/384; 435/307.1; 435/284.1; 435/1.3; 435/374; 435/420; 977/902; 977/773

(57) ABSTRACT

A system for cryoencapsulating a natural or manmade biological specimen which is capable of enabling the nucleation of at least one of benign polycrystalline or vitreous ice in the specimen via the introduction of particulate nuclei and cryoprotectant into the specimen including: (a) one or more specimen cooling modules or stations; (b) one or more specimen particulate or particulate-former introduction modules or stations; (c) one or more specimen cryoprotectant-introduction modules or stations; and (d) a system control unit and necessary system utilities, wherein the system is to implement a combined process of specimen cooling, specimen particulate introduction or formation, and specimen cryoprotectant introduction, and wherein the introduced particulate is to favorably enable the nucleation of one or both of encapsulating polycrystalline or vitreous ice resulting in ice formation with at least one of reduced ice damage or cryoprotectant toxic damage to the specimen during cryoencapsulation or during eventual thawing relative to a particle-free process.

### BACKGROUND

Current cryopreservation technology provides an attractive means of storing biological materials that have small masses for extended periods of time in a suspended or substantially arrested state of metabolism. Many medical, agricultural and biological applications are practiced such as the preservation of bodily fluids, bodily tissues and plant seeds. Cryopreservation allows for building a storage bank of such preserved materials such that it can be used on-demand in medical emergencies, future medical procedures, or to avoid extinction of threatened species. Historically, only small tissue masses have been cryopreservable with tolerable levels of ice and/or cryoprotectant damage. The reason is simple: small samples can be cooled throughout more quickly than large samples. The invention herein teaches a new and specific cold-encapsulation material, method and system to avoid these damaging phenomena and therefore allow for the preservation of much larger tissue masses such as bodily organs. The invention should allow more time for better typing of organs before transplant and thereby lead to a higher number of successful organ matches being made. It should allow more time for the transport of organs and more time for the transport of transplant recipients, which will increase patient access to donor organs. The invention also allows for reduced mechanical stress in cryopreserved parts, preservation-related stress being a serious safety and yield issue for the preservation of tissues such as arteries, veins and hearts. Unlike the prior art, this invention results in preserved encapsulated tissues with a specific protective structure which contributes to successful cold-preservation.

By “cryocooling I mean a specimen is cooled and stored at a temperature well below 0 Deg C., most conveniently and preferably at liquid nitrogen temperatures of around −196 Deg C. where at measurable metabolism is halted. There are three methods known for cryopreservation, but the first one discussed below, “slow-cooling”, is the one that currently is in wide use on a commercial basis for small-mass specimens.

“Slow Cooling” Cryopreservation: In this commercially, well established approach small amounts of cellular matter or tissue are slowly cooled or refrigerated at a rate of roughly 1 Deg C./minute, typically in the presence of a cell-permeating cryoprotectant(s). The purpose of the cryoprotectant(s) (an antifreeze, really) is, via osmosis, to replace some or all of the unbound intracellular water in the cells of the specimen. In that manner formation of destructive macroscopic ice crystals in the cells is substantially avoided or limited. Uncontrolled growth of large and faceted intracellular ice crystals is known to be fatal to cells. Further, the cryoprotectant(s) prevents and/or delays some extracellular ice formation, particularly as the intracellular water is leached out of cells into the extracellular space via osmosis. Disadvantages of the commercialized slow-cooling approach include the following:
i) cells experience high concentrations of and extended exposure to toxic solute and cryoprotectant in their interiors as water slowly leaves via osmosis;
ii) cells experience toxic high-cryoprotectant exposure on their outsides and permeating into their membrane walls; and
iii) some large (compared to the cell size) intracellular ice crystals still form which mechanically can disrupt cell membranes or damage intracellular organelles.

So, slow-cooling processes are optimized to trade-off freezing damage (intracellular crystalline ice-crystal disruption) against toxic-exposure damage from cryoprotectant such that some useful fraction of the preserved cellular matter usually survives in small specimens. Blood, semen, human eggs, corneas, heart valves and human skin are among tissues or cells that are preserved commercially using slow cooling. A host of software-programmable refrigeration/freezing equipment is available for this purpose. Typically the cells are thereby refrigerator-frozen to −30 to −50 Deg C. or so and then quickly immersed in or exposed to the effects of liquid nitrogen for long-term storage at −196 deg C. The process is used mostly on physically small and/or thin specimens which can undergo the aforementioned refrigeration cooling rate uniformly in a total time that is short enough to avoid massive intracellular ice formation.

“Rapid Cooling” Cryopreservation: Rapid-cooling cryopreservation involves hugely faster cooling rates such as hundreds or thousands of degrees Centigrade per minute i.e., 1000 Deg C./min or even faster by quench-cooling or splat-cooling and approaching 1000 Deg C./second or higher. This is so fast that the water in cells containing permeating cryoprotectant vitrifies (forms glassy amorphous ice) rather than crystallizing as orderly crystalline ice. There simply isn't enough time for the water molecules to rearrange into orderly crystals at these extremely high freezing rates, and rapid achievement of low temperatures also beneficially slows the mobility of the water (and cryoprotectant) molecules via a viscosity increase. Making this process work requires very high cryoprotectant concentrations to force vitrification, and these concentrations are even more toxic than those used for slow-cooling processes. Extracellular water in the specimen also substantially vitrifies. Even pure water can be vitrified at extraordinarily high cooling rates, but cryoprotectants usually are used today for actual cellular matter of significant mass such as an artery. Disadvantages of vitrification are
i) the desired huge cooling rate is virtually impossible to attain in anything other than a tiny or thin sample such as sperm;
ii) the toxicity of the higher cryoprotectant concentration kills or damages a large portion of cells despite the shorter exposures at intermediate temperatures; and
iii) devitrification (the growth of ice crystals from amorphous ice) can occur upon rewarming, thereby causing disruptive mechanical cellular damage similar to that experienced historically in early “slow cooling” development.

“High Pressure” Cryopreservation: High-pressure cryopreservation is a highly experimental and more recent process that involves the mechanical suppression of the freezing temperature of the specimen via the application of high mechanical pressures on the order of hundreds to thousands of atmospheres. These are as large as the geological pressures occurring deep within the earth. The known physical effect of such pressure applied to water depresses the formation of ice to temperatures well below 0 Deg C. and results in different crystalline ice structures than ice formed at normal atmospheric pressure. Ice formed in this manner has a density higher than water rather than lower than water. The freezing point of water drops about 0.55 Deg C. per 80 atmospheres of positive, applied incremental pressure. The idea is that the specimen is supercooled to as much as 10 to 23 Deg C. below the 1-atm freezing temperature (normally about 0 Deg C. at 1 atmosphere) while applying thousands of atmospheres pressure before the water transforms from liquid to ice. The main advantage is that the specimen reaches a lower temperature before freezing at the suppressed freezing point, and when freezing does occur it takes place more rapidly resulting in somewhat smaller-size ice crystals than slow-cooling methods. Also, some of the cryoprotectant toxicity effects are reduced because more of the toxic exposure occurs at lower temperatures. The technique has significant disadvantages which include:
i) the required pressure vessels are expensive and complicated and present operator-safety concerns, particularly since the required very low temperatures may cause brittle cold-fracture of the metallic pressure vessel, plumbing and sensors, and
ii) there are some newly introduced pressure-induced damage effects to the specimen which need characterization for at least partial avoidance.

Not surprisingly then, slow-cooling with cryoprotectants is the predominantly used process because it utilizes workable, easily operated, programmable-refrigeration means to get the temperature well below 0 Deg C. (into the −40 to −80 Deg C. range, for example) followed by a rapid physical plunge into liquid nitrogen to −196 Deg C. The severe limitation is the small-mass requirement for specimens that can be preserved successfully.

None of these three processes to date, however, has been demonstrated capable of cryopreserving whole organs because such specimens involve much thicker tissues across which it takes increasing amounts of time to extract heat and/or permeate cryoprotectants inward. This results in not being able to remain in a workable process window of toxicity damage vs. ice damage for the entire depth or thickness of the specimen as the freeze-front penetrates through the specimen over time. Although the mentioned high-pressure techniques might improve the situation, there is a considerable hurdle regarding the hardware involved, and long-term storage at super-high pressure would be expensive and could invite even more cell-damage mechanisms to be concerned about. Further, routine access to the specimen(s) might be inhibited if one cannot relax and reapply pressure multiple times, such as if a portion of a preserved sample is utilized over a period of time or if a bit of the preserved sample must be removed to perform tissue matching or cell typing.

### BRIEF DESCRIPTION OF THE DRAWING

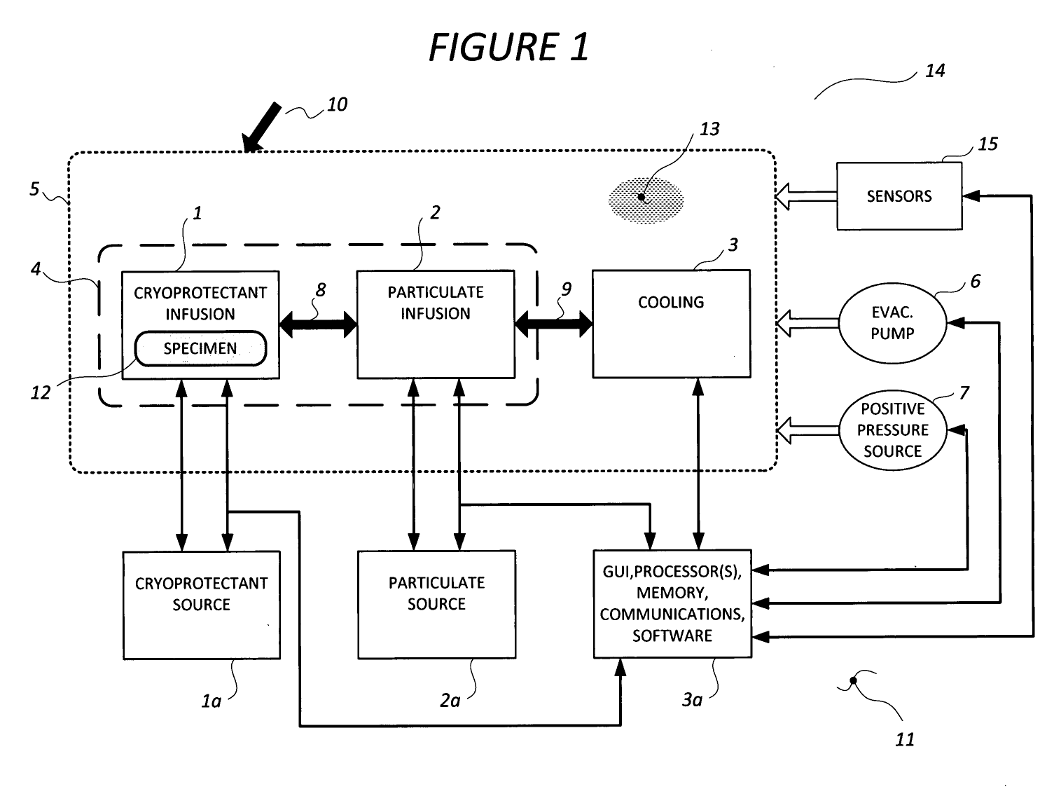

The sole FIGURE, FIG. 1, is a schematic drawing of a cold-encapsulation system in accordance with an embodiment of the invention.

### DETAILED DESCRIPTION

Applicant views the invention as a fundamentally new tool with which to move beyond existing cryopreservation techniques into more advantageous or more forgiving ice-damage vs. toxicity-damage tradeoffs or to develop entirely new cold-preservation processes. In some cases Applicant anticipates the invention allowing the complete avoidance of all significant ice damage and/or toxicity effects and not just the reduction thereof.

Applicant does not take the conventional view that appreciable intracellular crystalline-ice formation, per se, always is fatal to cells. While Applicant agrees that slow-cooled large ice crystals with macroscopic facets and edges usually are fatal because they puncture cell membranes and likely rupture cell organelles, Applicant believes that cells, especially with some amount of cryoprotectant, could accommodate large numbers of tiny or small (compared to cell diameter) ice crystals which form on engineered nucleation sites or on preformed nanoparticles that are delivered into cells to act as such nuclei. In one embodiment, an array of tiny ice crystals, likely forming at least some merged polycrystalline ice or close-proximity microcrystal groups, is formed in the cells. Because the individual ice crystals are so tiny and because their forgiving polyhedral shape is encouraged by the crystal-growth drag effects of the cryoprotectant, they can be formed without puncturing the membrane and they can, to a significant degree, spatially nucleate and grow in a manner that accommodates the cell organelles and other intracellular structures. Given this, the process may use less cryoprotectant, commensurate with the need to extract less cellular water, thereby reducing cryoprotectant-toxicity effects and membrane-collapse effects. Further, huge vitrification-style cooling rates are not necessarily needed as polycrystalline ice or finely dispersed microcrystals become acceptable over bulk vitrified ice. By “tiny” ice crystals I mean capable of forming around organelles within the cell membrane. Such crystals would in some cases have average sizes of from 0.1 D down to 0.001 D, where D is the characteristic diameter of the interior of the cell. This allows for many microcrystals in a given cell, whether they are arranged in isolation, close proximity, or in abutted or fused polycrystalline clusters. This is accomplished using controlled, simultaneous nucleation of multiple microcrystals, each of which has its crystal growth slowed by the interfering effects of cryoprotectant, which not only increases viscosity but also can be chosen to interfere with or slow crystal growth. Further, Applicant anticipates embodiments wherein, because there are so many microcrystals nucleating and growing in each cell, that their competing growth physically limits their maximum size within the confined space of the cell. The inventive controlled nucleation may also—or instead—be practiced in the extracellular spaces as opposed to the intracellular spaces.

Vitrified ice also may exist. For macroscopic samples such as organs it may be unavoidable that different regions of the organ (e.g. outer surfaces vs. deeply internal portions) end up with different inventive ice structures (e.g. vitreous vs. polycrystalline or polycrystalline grain size 1 vs. polycrystalline grain size 2) but all of the preserved structures present are undamaging and nontoxic in nature, and all such ice crystals are very tiny and accommodating of the cell and its contents. This approach allows more preservation-process bandwidth for organs because ice-crystal growth no longer is fatal, and a large organ could have a vitreous extracellular space and a microcrystalline or nanocrystalline intracellular space, for example. Alternatively, a large specimen could have only nanocrystalline ice everywhere or vitreous ice everywhere. This is because the inventive ultradense array of nucleation sites allows the formation of either superfine ice crystals and/or vitrification to be accomplished at a slower cooling rate at a given cryoprotectant concentration.

Another general aspect of prior-art cryopreservation which many consider to be a fundamental limit are damaging macroscopic stresses which may prohibit one or both of:
i) practical whole-organ preservation with any yield, or
ii) whole-organ preservation with good yields (e.g., 85% success rate into a cold-preserved state, 95% for the ensuing thaw using current estimates for transplant success).

While this may be true of the existing technologies and combinations thereof, Applicant claims a new paradigm. Specifically, if the cells themselves can be preserved (such as with Applicant's nucleation-controlled polycrystalline and/or vitreous ice formation), then one no longer is locked into high ice content in the extracellular space, i.e. surrounding the cells. One also succeeds in widening the tradeoff window between ice damage and toxic damage.

Applicant claims an encapsulating preservation technique wherein the cells themselves are preserved using either or both of fine-crystal or vitreous ice nucleated by the inventive particulate combined with a much lower ice-content extracellular matrix material which retains some plasticity or creep allowance (stress relaxation) even at subzero temperatures. This can be done by doping the extracellular space with cold-compliant materials such as polymers, polymer formers, or nucleated gas bubbles which do not easily penetrate the cell membranes. The cells themselves already might be preserved at this point. Thus the extracellular structure can be quite different from the intracellular structure. The result is a somewhat ductile cold-preserved organ which will not stress-crack or shatter upon impact even at subzero temperatures. This is unthinkable with the current cryopreservation tools. They all cause large concentrations of brittle extracellular ice.

The invention comprises the purposeful creation or provision of a densely dispersed fine particulate, in some cases at least inside the intracellular space of cellular matter to be protected. This particulate acts to nucleate ultrafine crystalline-ice formation or vitrified-ice formation at a far greater number and density of nucleation sites than current processes.

The particulate is one or more of a physical particle (solid or liquid or gas). In the case of gas “particulate”, the gas may be provided as an array of microbubbles or nanobubbles formed (nucleated) upon a sudden reduction of an applied pressure, for example. In the case of a solid or liquid particulate, the particulate material(s) could include, for example, administered or injected nanoparticles or a chemical or phase-change precipitate formed in-situ. In all cases the particulate either (i) triggers ice formation (crystalline or vitreous depending on temperature) directly upon or adjacent to itself, or (ii) triggers nucleation of another material directly upon it which in turn then has ice (crystalline or vitreous) nucleate upon that dual-material nucleus. So the particulate directly or indirectly nucleates crystalline ice at each particle or eases the formation of vitreous ice in particle-laden fluid and tissue. Cryoprotectant(s) additionally can suppress or slow crystalline ice growth or encourage vitreous ice formation, and some appreciable amount of cryoprotectant can be adsorbed and have its toxicity isolated by polycrystalline ice-grain boundaries or particle-laden vitreous ice.

The invention may utilize known cell- and/or membrane-permeating cryoprotectant(s) for intracellular and membrane preservation and slow and/or rapid cooling. However, Applicant's dispersed particulate itself acts as a cryoprotectant because of its favorable manipulation of safe crystalline or vitreous ice formation and toxics distribution.

The invention also may use nonpenetrating extracellular “ductile matrix material” which does not penetrate cells but does penetrate extracellular space.

The invention also may utilize extremely high pressure (e.g., 1000-2000 atmospheres) to shift freezing points lower mechanically but more often will utilize much lower pressures, e.g., a few atmospheres, for purposes of triggering or suppressing nucleation of microbubbles and/or ice nuclei.

An inventive system, whether assembled into a single structure or provided as separate modules or process stations, schematically would include the following:
a) A specimen cryoprotectant-introduction module.
b) A specimen particulate-introduction module.
c) A specimen-cooling module.
d) A specimen-pressurization/depressurization module if phase-transformation temperatures are manipulated using pressure or if gaseous-particle nucleation via pressure change is practiced.
e) Supporting utilities and hardware as required for refrigeration, cryogenic cooling, cryoprotectant storage and flow, coolant storage and flow, temperature and pressure monitoring and control, user interface, power supplies, software, processor(s), memory and specimen monitoring sensors.

Included in the inventive scope is the application of sonic or ultrasonic shockwaves and/or abrupt pressure changes produced using transducers such as piezoelectric transducers for manipulating nucleation behaviors and/or assuring that cryoprotectants can penetrate membranes. The transducers would trigger the particulate-aided ice phase change(s). Transducers may be used in addition to more conventional hydrostatic-pressurization means such as hydraulic pumps and compressed-gas pressurization or actuation. Shockwaves have the potentially attractive attributes of being directionally controllable such that induced nucleation can propagate in a desired direction and of being able to cause cavitation if that is desirable for nucleation from introduced particulate (e.g., gold or iron oxide nanoparticles) or particulate-forming material (a dissolved gas).

Before proceeding I will define the terms used herein.

Definitions:

Conventional Cryoprotectant: Any substance placed in physical contact with or introduced within specimens to be cryopreserved (or inventively cryoencapsulated) which reduces, avoids, delays or suppresses a degree of ice damage due to one or both of actual or potential crystalline ice and/or vitreous ice formation. Conventional cryoprotectants include but are not limited to, for example, ethylene glycol, propylene glycol, glycerol, glycerin, DMSO (dimethylsulfoxide), 1,2-propanediol, methanol, trehalose and other sugars. Most often, mixtures of two or more such cryoprotectants are employed. These cryoprotectants typically are introduced by liquid immersion, irrigation or perfusing permeation. Other additives sometimes are used to reduce cryoprotectant toxicity, chill injury or denaturing injury, and the invention herein may utilize any such cryoprotectants and additives as are known or discovered.

Particulate Cryoprotectant: Any solid, semisolid, liquid or gas material introduced into the specimen, such as by dissolving, injecting, entraining, perfusing or precipitating for the inventive purpose of at least one of:
a) hosting or encouraging the formation of crystalline and/or vitreous ice at, on or in the dispersed particles;
b) interfering with or suppressing crystalline- or vitreous-ice formation when the particles reduce the mobility of water molecules or interfere with orderly crystal growth at the atomic level; or
c) utilizing a molecularly targeted cryoprotectant particle or particle-molecule to attach to specific sites on protectable specimen structures.

At first it may seem counterintuitive that the action of (a) can act to cryoprotect. However, it can do so in situations wherein:
i) a very fine, accommodating, polycrystalline ice is formed which minimizes physical puncture or tearing damage to cellular matter and/or may be mechanically stronger than coarse ice or vitreous ice so that specimen breakage is avoided;
ii) a polycrystalline ice is formed which has the ability to “soak-up” and isolate conventional cryoprotectants in their numerous grain boundaries, thereby reducing toxic damage;
iii) ice (polycrystalline or vitreous) is formed which contains microbubbles or nanobubbles (from microbubble- or nanobubble-particulate nuclei) which allow ice to creep more easily and relieve thermally induced stress;
iv) the “particulate” cryoprotectant is actually a precipitating cryoprotectant or a precipitating constituent thereof; thus, particulate precipitation may occur from a liquid cryoprotectant, for example;
v) fine polycrystalline ice formation is more forgiving to the specimen's intracellular entities and can, to a significant degree, form around them in a mechanically less-damaging manner;
vi) the specific particulate suppresses a freezing point (crystalline or vitreous) in a chemical or physical manner;
vii) the particulate nourishes cells or scavenges highly reactive radicals;
viii) the particulate allows for a controlled, global ice-nucleation event such as the virtually instantaneous creation of dispersed microbubbles of gas via a modest applied-pressure reduction upon a gas saturated specimen; or
ix) a combination of fine-grained polycrystalline ice and vitreous ice are formed, possibly intermixed, one or both possibly having suppressed formation temperatures due to particulate presence.

The unrelated use of nanoparticles and microparticles for purposes of targeted drug delivery or targeted gene therapy is known. Many of these utilize relatively inert particulate solids such as gold or iron oxide and others utilize liposomes which are organic in basis or biological in nature. The particles are modified, such as by the attachment of surface molecules, so that they can affix onto target molecules in the patient. Other particle modifications could include loading a hollow liposome particle with a drug payload or a contrast agent. Other such particles are being researched for use as ultrasound contrast agents, which also may carry drugs or target cancer. Applicant's invention may utilize any such particle as long as it nucleates or causes the nucleation of either a bubble or a local ice-formation event. The particles may have these known abilities as well.

The inventive particles may be targeted to specific specimen targets or, for example, alternatively might be targeted molecularly to specific specimen cellular targets such as organelle targets or cell-membrane targets. In all cases, particles could be introduced globally as by specimen immersion, perfusion or diffusion, in-situ precipitation or nucleation. These particles may comprise microparticles or nanoparticles or may consist of single, targeted, cryoprotectant molecules per the above definition.

Within the inventive scope are particulates comprising nucleated (such as by an abrupt pressure reduction) bubbles or acoustically cavitated bubbles. Such bubbles may then either (or both) serve to nucleate ice formation or serve to allow creep and to relax thermal stress.

### Examples of Particulate Cryoprotectants:

### Solid and Semisolid Particles

Microparticles and nanoparticles smaller than about 7 microns in size can pass through tissue capillary beds. Microparticles have an order-of-magnitude size measured in microns whereas nanoparticles have an order-of-magnitude size measured in nanometers. Thus, one can introduce such particles into tissue such as by flowing them through arteries and/or veins. To some degree they can also attain extracellular and/or intracellular positions as by diffusion if they are small enough or have shapes which can pass through cell membranes, as can known proteins and sugars, for example. Known solid particles which are biocompatible include gold and iron oxide nanoparticles and microparticles. Nanoparticles can diffuse through any biological material. They can be engineered (e.g., molecularly-targeted) to attach preferentially to specific biological structures. In this manner one might assure specific cryoencapsulation protection for specific organelle or membrane entities, for example. Included in the inventive scope is the use of targeted molecules whose purpose is to assure cryoprotection of a targeted structure in a cell. That protection could involve the triggering of local nucleation or simply the targeted delivery of a cryoprotectant molecule(s) that is optimized to preserve a specific structure.

### Entrained Gas Particles or Microbubbles/Nanobubbles

Clusters of gas molecules or micro/nano bubbles can be entrained in many liquids. This gas is not dissolved but exists as clusters of atoms or molecules. It is not dissolved for one of several reasons, such as:
a) the entrained gas is gaseous above the solubility limit of the gas, i.e., the undissolvable portion;
b) the gas has poor mobility in the liquid so that larger-bubble growth is suppressed; or
c) the gas is surrounded by a surfactant or coating which stabilizes the microbubble or cluster of microbubbles.
   Within the inventive scope is the use of surfactants to manage bubble or particulate stability or formation or to manage the stability of cryoprotectant solutions.
   Nucleated Gas Microbubbles from Dissolved Solution

Many gases are soluble in one or more of tissue, blood and cryoprotectants. These include nitrogen, oxygen (usually tied up with hemoglobin if it is present and unsaturated), helium, carbon dioxide, argon, neon and hydrogen. The solution/dissolution behavior of these has been well characterized for safe diving purposes by the US Navy, as have the negative effects such as oxygen narcosis. However, for the preservation of organs such as a heart or kidney, many of these negative effects on living beings do not apply to excised organs and the invention herein purposely involves the nucleation of microbubbles or nanobubbles of such gases to act as the inventive (gaseous) particulate cryoprotectants. Such gases can be introduced into tissue specimens such as by pressurization in the gas or by immersion/flushing/perfusion/irrigation in a liquid containing the concentrated or saturated gas. This approach frequently will involve a pressure chamber capable of at least a few or several atmospheres of pressure application for at least the gas-permeation or saturation step. Abrupt pressure decreases can cause sudden fine-scale nucleation of such microbubbles or nanobubbles. Such exposure may be upon the organ/tissue after removal or may be upon an entire donor patient such as before organ removal. An abrupt pressure change may be applied as by pressure-venting or by the application of an acoustic shockwave. If the shockwave-nucleated bubbles are simply exsolution bubbles (gas coming out of solution) then true cavitation is unnecessary. An acoustic shockwave has the advantage of extremely rapid application and directional travel. A specific preferred approach to cryoencapsulation is that wherein suppression of a particulate and/or a pressure-induced phase transition allows a dense array of particulate to nucleate ice formation (crystalline or vitreous) densely and at a high rate. This approach offers a path to avoid formation of large ice crystals.

### Organic Microparticles or Nanoparticles

These include liposomes either with or without interior cavities, for example, cavities filled with a gas such as a perfluorocarbon or fluorocarbon. The invention is intended possibly to utilize the vast storehouse of organic nanoparticles and microparticles that are being developed for targeted drug delivery, targeted therapy, targeted gene therapy and targeted imaging contrast agents. “Targeted” means the particle has a biological target in the tissue such as a cell membrane, cellular DNA or a cell interior structure or organelle. Thus, the inventive cryoprotectant might be targeted to attach to specific cell or tissue portions.

### Penetrating and Nonpenetrating Cryoprotectants:

By penetrating I mean substantially penetrating intracellular space, at a concentration that is close or equal to the concentration in the extracellular space. By nonpenetrating I mean not substantially penetrating intracellular space, or far below the concentration of cryoprotectant outside the cell. The cells in question may or may not themselves be cold-preserved at the time of this penetration attempt. Thus, nonpenetrating cryoprotectant doesn't penetrate cells under at least one circumstance such as not penetrating unprocessed cells or, alternatively, not penetrating an already preserved cell. This distinction is made because it may be only cells that are already preserved which are not penetrated by particular nonpenetrating cryoprotectants. Note, therefore, that a cryoprotectant which penetrates extracellular space still may be nonpenetrating for intracellular space. By substantially penetrating I mean completely or nearly completely penetrating.

### Ductile Matrix Material or “Ductile Cold Preservant”

A material which substantially penetrates extracellular space and offers some amount of global cold ductility (brittle-fracture resistance) even at subzero temperatures. The material itself may offer prior-art cryoprotectant attributes or it may be doped with or mixed with some cryoprotectant offering such known attributes. Applicant regards this material as cold-preserving because it at least causes the specimen to retain significant mechanical toughness, even if it is chosen to have no other prior known benefit. This material also may offer stress relief, which also decreases fracture behavior or tendency. Applicant notes that the extracellular space forms a three dimensional, connected matrix around the cells and thereby can offer global ductility even if individual cells embedded therein are inherently brittle.

### Cryoencapsulation:

The cold preserving of biological specimens in inventive particle-seeded structures utilizing inventive particulate technology wherein at least some or both of intracellular space and extracellular space is occupied by a solidified frozen material which solidifies under the influence of or in the presence of a dispersed introduced particulate, the particulate improving the preserving process. The particulate will be present in the specimen at least long enough to influence the ice-nucleation behavior favorably. In many cases, such as for solid micro or nanoparticles, the particles will remain in the ice indefinitely. It is key to the invention herein to recognize that any crystalline ice formed can be finer grained than the prior art due to the millions of densely competing nucleation events and that vitreous ice formed by the invention can form at lower temperatures than by the prior art.

### Discussion of FIG. 1

Inventor provides FIG. 1, which schematically depicts an inventive cold-encapsulation system 14. The major process modules and utilities, presuming for clarity that they are individualized and not shared, are a cryoprotectant-infusion module 1 serviced by a cryoprotectant source la, a particulate-infusion module 2 and supporting source of particulate 2a, a cooling module 3 likely including both refrigeration and cryogenic cooling hardware as required, and some control utilities 3a. Inventor has stated that some modules may be combined physically and two such possibilities are depicted by the dashed lines 4 and 5. Specifically, dashed line 4 indicates that cryoprotectant-infusion module 1 and particulate-infusion module 2 may form a common module or chamber 4 or may be physically connected. As a further possibility, dashed line 5 shows the case wherein all three of the modules 1, 2 and 3 are the same module or chamber or are closely connected modules or chambers.

Looking at FIG. 1, it is seen that the inventive cold-encapsulation system 14 is placed in a room or lab ambient 11 which will usually be at one atmosphere pressure and approximately 25 Deg C. A specimen 12 is depicted as sitting in a dedicated cryoprotectant-infusion module 1. Dual arrows such as arrows 8 and 9 show that for a true multimodule design, the specimen 12 would be moved or transported among the various chambers, perhaps even several times, and perhaps in one or both directions. Such movement or transport 8, 9 between chambers or modules could be in one or more of several ways including such as:
a) Manual transfer in a protective container (container not shown);
b) Automated transfer in a protective container (container nor shown);
c) Manual transfer while exposed to the ambient (the ambient may be sterile or clean);
d) Automated transfer while exposed to the ambient; or
e) Transfer on an interior or exterior track or conveyor or though an extended intermediate chamber passage (not shown).

By “protective container” Applicant means a specimen-transfer container that protects against one or more of contamination, outward diffusion of a cryoprotectant or particulate, inward diffusion of air, undesirable warming or undesirable condensation of ambient moisture which turns to frost. It might also mean transfer at a controlled temperature and/or pressure within the container, thereby protecting from contamination by human touch or prevention of user injury due to potential cold or chemical exposure. Presuming a transfer is of a specimen not yet cooled but which has been inwardly diffused with cryoprotectant and/or particulate, the transfer container might be thermally uninsulated at one atmosphere, which simply assures that the specimen remains submerged in saturated cryoprotectant. The container, if employed, may itself serve as a pedestal for the specimen as the specimen moves through each module, i.e. the specimen remains in the container from start to finish even if the container with specimen moves through the system.

An exemplary implementation is that indicated such as by dotted line 5 wherein all of the cold-encapsulation subprocesses to be implemented upon the specimen 12 can take place in one module or chamber or pressure-vessel tank without transfer of the specimen 8, 9 outside the common module or chamber. Further, in that case, utilities 1a and 2a also might be combined with each other and together with entity 5 (option not shown) especially if the cryoprotectant is delivered to the specimen with the particulate material already dissolved or entrained in it. The positive-pressure source 7, as noted previously, could apply pressure for the purpose(s) of lowering a freezing point of water and/or of a cryoprotectant or could be employed to reduce or bleed a prior-applied pressure in order to cause supersaturation for causing particulate nucleation, for example. For safety reasons it may be desirable to pressurize only solids and liquids, which have very low compressibilities compared to gases, because in case of a pressure-vessel or pressure-hardware failure the explosion will be of low energy and minimal violence. That consideration would lead one to fill completely any chamber to be highly pressurized for any reason with liquid cryoprotectant solution, for example, and utilize a hydraulic pump to apply hydraulic pressure via such as an isolation diaphragm. If a liquid cryoprotectant must have predissolved particulate or particulate-forming material dissolved in it to high concentrations, then that combined liquid might be presented as chilled liquid (maximal-solubility, particulate-forming gaseous material) or as gas-pressurized liquid that is perhaps at a few tens of PSI, but with a small gas volume to minimize explosive energy potential.

If modules 1, 2 and 3 all share a common chamber or are actually the same shared module or chamber (the dotted line 5 option), I show both an evacuation pump 6 and a pressurization pump 7. Evacuation pump 6 would be such as for reaching sub-ambient atmospheric pressure if that is used in the specific overall process, such as to remove air from a chamber or to outgas air or other gas from the specimen or cryoprotectants. Pressurization pump or positive-pressure source 7 would be employed to pressurize the specimen/cryoprotectants/particulate such as to depress a freezing point or to release some of said positive pressure to achieve supersaturation of a particulate-forming material such as of a gas to nucleate microbubbles. One might also encourage supersaturation of a particle-forming gas by using the evacuation pump to drop below an ambient pressure previously maintained over a gas-saturated cryoprotectant. However, it is faster to relieve pressure by bleeding or venting than by pumping. Of course, electronically controlled mass flow-controllers or throttling valves could make precise handling of flows, pressurizations and depressurizations highly repeatable and capable of being automated.

Sensors are depicted as items 15 and these may include, for example, temperature and pressure sensors, specimen-location sensors, fluid or gas presence/amount sensors, ice-formation sensors, nucleated- or precipitated-particulate sensors, compositional sensors, pH sensors, and dissolved-gas sensors. Additional sensors which look at dissolved ions such as potassium and sodium (the often employed K+/Na+ viability ratio) also are known to the field and are known to indicate a good freezing process. Inventor also anticipates the employment of optical coherence tomography (OCT) using a fiber inserted into the sample and passing through a chamber feedthrough. Such an OCT device is capable of imaging ice nucleation, ice growth and particulate nucleation, and such a device could be inserted safely into the core of the organ specimen without permanent unacceptable damage to the organ. OCT and ultrasound therefore may one of or both be employed to image or detect such phenomena that the system is attempting to bring about and control. Ultrasound or ultrasound imaging also may be employed to monitor the ice growth process.

The system is controlled and operated via the controls portion 3a which also includes a control panel and likely a graphical display. This portion may be interconnected to all of the other modules, instruments and utilities as depicted such as by power, signal and data wires and gas/liquid plumbing. In that manner, an operator and/or system software can coordinate the operation of the various functions and utilize sensor feedback in doing so. The reader is reminded that the particulate may be introduced conveniently into a specimen 12 after the particulate has already been suspended, entrained-in or dissolved-in a cryoprotectant liquid; i.e. a mixture of cryoprotectant and particulate is presented to the specimen. In that case, some or all of modules 1 and 2, if not also utilities 1a and 2a, may be combined physically.

An embodiment of a system design has the specimen sitting, held or immersed in a fixed location during all of cryoprotectant infusion, particulate infusion and cooling. This could be one combined chamber or module such as 5 with copackaged utilities 1a, 2a and 3a as well, for example. Inventor stresses that the system will, for some processes, undertake two or more subprocess steps simultaneously such as cooling and cryoprotectant infusion, cooling and mixed cryoprotectant/particulate infusion or even cooling and cryoprotectant infusion and particulate infusion. Further, during such single or combined processes the pressure may be varied such as to reduce a freezing point or to encourage nucleation or precipitation of particulate nuclei. Automated operation is advised particularly if more than one variable is changed simultaneously.

Arrows 8 and 9 show that in some system implementations the specimen may be moved between modules or moved from one process station or chamber to another in a single combined system. Regardless of the specific implementation, the specimen will enter the system at some physical location such as 10 which might comprise a sealed lid, cover or air-lock, for example. It may exit from the same or a different (not shown) port or unload location.

Presuming, again for discussion purposes, a shared module 1, 2 and 3 forming module 5, a depiction of material or chamber ambient 13 is shown filling the shared common chamber of module 5. During cryoprotectant infusion, particularly for liquid cryoprotectants, inventor expects the specimen 12 to be completely immersed in liquid cryoprotectant(s). In that case, item 13 likely would be liquid-cryoprotectant filled to at least a specimen-submerging level if not filling the module chamber 5 completely so as to eliminate free compressible gas in space 13. In the case wherein a cryoprotectant predissolves or pre-entrains a nucleatable particulate material, it is likely that the cryoprotectant would be pressurized (and/or cooled) to increase the solubility of the nucleating materials. In that case, cryoprotectant source 1a might apply pressurization to the cryoprotectant/particulate nucleator even before the cryoprotectant is introduced to the specimen 12.

In a different scenario, the cryoprotectant and nucleatable material first are introduced physically to the specimen 12 and then pressurized as by pressurization source 7.

The depicted system 14 is a flexible system capable of any process sequence that can be programmed or requested by a user. The system may process one or more specimens at a time (as separate or batched specimens) and in some scenarios the system could operate in a fully automated manner. Because of the multiple feedback sensors, one could use the system to conduct a set of experiments to optimize a cold-storage process knowing that all of the settings were precisely controlled and accurately monitored. Researchers in the cryopreservation field have been hampered by the marginal reproducibility of manually conducted experiments from lab to lab and even from day to day within a single lab.

The system 14 may have an exit port (not shown) which essentially automatically emits the preserved sample into a collection container or room which comprises protective storage such as into a liquid-nitrogen-cooled vat or packaging.

Depending on the number of modules employed and entry/exit ports used it may be beneficial to have the system employ load locks which are specimen 12 entry and exit subchambers (not shown) which isolate a main processing chamber from the atmosphere such that the main chamber can maintain a condition such as an applied positive pressure, a reduced pressure and/or temperature or filled with a liquid.

The system instead or also may employ a standardized holder or cassette to contain specimens, particularly if the same size or shape organ is processed repeatedly. Applicant includes within the inventive scope the use of a tissue phantom, which is a dummy specimen that has easily measurable phase changes and nucleation behavior and can be manufactured in repeatable fashion. In this manner, the phantom specimen can be used to validate process control presuming the mass and dimensions of the real specimen remain within an acceptable range. The phantom, depending on its size and thermal mass, would be run alone (no specimen), run together with the specimen, or run while embedded in a specimen. Its purpose is to verify an expected result given that the phantom has precisely known thermal properties if not also freezing and nucleation behaviors. It could be regarded as a “witness specimen” and it would verify lab-to-lab reproducibility independent of specimen-to-specimen variations.

The invention not only improves the ice-damage vs. toxicity tradeoffs but it also provides a mechanically superior, tougher cryoencapsulated specimen in many embodiments. The improved tradeoffs allow the widening of process margins that will be necessary to cryoencapsulate organ-sized specimens. Specimens incorporate polycrystalline and/or vitreous ice structures whose formation is nucleated by the inventive particulates in a manner providing one or both of less ice damage or less toxic damage. Finally, in some embodiments the extracellular space can be filled or infused with a ductile cryoprotectant providing superior mechanical toughness and stress relaxation.

The types of specimens which may be cryoencapsulated include at least the following types:
a) human or animal tissues or body fluids;
b) organs or biological tissues;
c) plants or plant derivatives;
d) food or food derivatives;
e) marine life or derivatives thereof;
f) seeds or pollen;
g) a tissue, fluid or material containing genetic information;
h) fruits or derivatives thereof;
i) vegetables or derivatives thereof;
j) body parts, tissues or fluids for intended transplant or reimplant; and
k) cellular matter, whether natural or human-engineered.

The invention allows for the more benign formation of ice, whether crystalline or vitreous, in terms of mechanical and toxic damage to the specimen. It also can provide a tougher, more fracture-resistant preserved specimen encapsulated within the inventive particle-laden ice and cryoprotectant material.

To current knowledge, at −196 Deg C., the temperature of liquid nitrogen, all measurable cell function and degradation processes cease. In the range of current refrigeration, −50 to −80 Deg C., it is not definitive that cellular degradation does not occur, particularly for longterm storage.

Therefore, it is likely that specimens prepared by the inventive system will be stored after cryoencapsulation at or near −196 Deg C.

For the majority of the embodiments taught herein, the inventive particulate causes the formation of benign crystalline or vitreous ice that is one or more of ice with a smaller crystalline grain size, fine polycrystalline ice which soaks up toxic cryoprotectant or ice with superior mechanical properties, or it is vitreous ice rather than crystalline ice. The particulate also may have inherent cryoprotective traits as well as, per the prior art, a property such as suppressing ice formation. For these the particulate may or may not be targeted to specific specimen target sites.

For an example case wherein the particulate is a single type of cryoprotective molecule with an attached, targeted, molecular-bonding species, that cryoprotectant molecule can be delivered and attached to a specific specimen target site that is intended to be protected. At that site, even if it does not encourage an ice-formation event, it still will offer presently unavailable, targeted cryoprotection. This allows use of less cryoprotectant and more efficient use of cryoprotectant, both of which will minimize cryoprotectant-toxicity damage. In this second protective mechanism, the particulate provides for benign ice formation by making sure that any ice formation, regardless of the ice type or properties, happens away from protected specimen targets.

What is claimed is:

1. A system for cryoencapsulating a natural or manmade biological specimen which is capable of enabling the nucleation of at least one of benign polycrystalline or vitreous ice in the specimen via the introduction of particulate nuclei and cryoprotectant into the specimen including:
a) one or more specimen cooling modules or stations;
b) one or more specimen particulate or particulate-former introduction modules or stations;
c) one or more specimen cryoprotectant-introduction modules or stations; and
d) a system control unit and necessary system utilities,
wherein the system is to implement a combined process of specimen cooling, specimen particulate introduction or formation, and specimen cryoprotectant introduction, and wherein the introduced particulate is to favorably enable the nucleation of one or both of encapsulating polycrystalline or vitreous ice resulting in ice formation with at least one of reduced ice damage or cryoprotectant toxic damage to the specimen during cryoencapsulation or during eventual thawing relative to a particle-free process.

2. The system of claim 1 wherein any of:
a) two or more modules or stations are physically joined, shared or common in nature;
b) two or more modules or stations are interconnected or intercommunicative in any manner;
c) two or more modules or stations are physically separated;
d) the specimen is supported in a specimen carrier or container;
e) the specimen is transported between modules or stations in any manner; or
f) the system utilizes process control sensors or a witness specimen.

3. The system of claim 1 wherein the system also includes a pressure-manipulation capability wherein at least one of a specimen, particulate or cryoprotectant can be exposed to a pressure change at least temporarily, said pressure change enabling one or more of:
a) the beneficial suppression of a phase-transition;
b) greater solubility of a particulate or particulate former; or
c) enablement of a subsequent pressure-reduction-induced particulate or ice-nucleation event.

4. The system of claim 1 wherein the system can perform one or more of:
a) introduction of precooled cryoprotectant to the specimen;
b) introduction of cryoprotectant to the specimen whereupon cooling is implemented upon both the specimen and the cryoprotectant; or
c) introduction of particulate or a particulate-forming material into the specimen either directly or via an introduced cryoprotectant.

5. The system of claim 1 wherein the particulate, as introduced into or formed in any of the specimen or cryoprotectant, comprises any one or more of solid, semisolid, liquid or gaseous particles, microbubbles, nanobubbles, acoustically-cavitated nanobubbles or microbubbles or targeted nanoparticulate or microparticulate.

6. The system of claim 5 wherein delivery of particulate or particulate-forming material to or into the specimen involves at least one of:
a) a particulate-containing or particulate-forming cryoprotectant;
b) particulate immersion, injection, diffusion, flushing or exposure; or
c) specimen infusion or flushing of a specimen lumen or organ cavity.

7. The system of claim 1 wherein the cooling modules(s) or station(s) include any one or more of:
a) a refrigeration capability to provide cooling to any one or more of a specimen, particulate or cryoprotectant; or
b) thermal exposure of any one or more of a specimen, particulate or cryoprotectant to liquid or liquid-vapor nitrogen thereby reducing the specimen temperature to approximately −196 Deg C. or below.

8. The system of claim 1 wherein the system is to infuse at least extracellular specimen spaces with a cold-ductile material, thereby creating a fracture-resistant and stress-relieving cryoencapsulated specimen.

9. The system of claim 1 wherein the particulate introduced into or formed in the specimen enables one or more of:
a) one or both of polycrystalline or vitreous ice formation or nucleation directly upon, in, at or adjacent to said particulates;
b) microbubble, nanobubble or acoustic-cavitational microbubble or nanobubble formation at or upon said particulates;
c) limitation of the maximum size of ice crystals;
d) reduction of the phase-change temperature of crystalline or vitreous ice; or
e) introduction into the specimen in a global or targeted manner.

10. The system of claim 1 wherein the system is to cryoencapsulate one or more of:
a) human or animal tissues or body fluids;
b) organs or biological tissues;
c) plants or plant derivatives;
d) food or food derivatives;
e) marine life or derivatives thereof;
f) seeds or pollen;
g) a tissue, fluid or material containing genetic information;
h) fruits or derivatives thereof;
i) vegetables or derivatives thereof;
j) body parts, tissues or fluids intended for transplant or reimplant; or
k) cellular matter, whether natural or human engineered.

11. A method of cryoencapsulating a natural or manmade biological specimen which encourages the nucleation of at least one of benign polycrystalline or vitreous ice in the specimen via the introduction of particulate nuclei and cryoprotectant into the specimen, said method comprising:
a) utilizing at least one of a refrigeration means or a liquified cryogen to directly or indirectly cool the specimen;
b) introducing a cryoprotectant into the specimen; and
c) introducing or forming in-situ particulates within the specimen by specimen exposure to a particulate or particulate-forming material, and the particulate in the specimen contributing to at least one of:
i) minimizing a size of ice crystals;
ii) promoting the formation of or maintenance of vitreous ice rather than crystalline ice;
iii) promoting the formation of or maintenance of fine-grained crystalline ice which has a large specific-surface area (grain-boundary surface-to-volume measure) with which to incorporate a cryoprotectant and thereby offer toxic protection therefrom;
iv) promoting the formation of a reduced-stress or stress-relaxing frozen structure;
v) minimizing ice-induced mechanical damage to the specimen;
vi) reducing the temperature of a crystalline- or vitreous-ice formation event;
vii) suppressing an ice-related phase change; or
viii) providing targeted protection of specific specimen portions.

12. The cryoencapsulating method of claim 11 wherein the particulate includes at least one of microbubbles, nanobubbles, acoustically cavitated microbubbles or nanobubbles.

13. The cryoencapsulating method of claim 11 wherein the particulate includes solid, semisolid, liquid or gaseous particles or molecules that are targeted to attach to specific specimen portions.

14. The cryoencapsulating method of claim 11 wherein particulate or particulate-forming material is at least temporarily contained in a cryoprotectant material.

15. The cryoencapsulation method of claim 11 wherein gaseous or vaporous particulate is formed via reduction or relief of an applied pressure.

16. The cryoencapsulation method of claim 11 wherein ice crystals of a size significantly smaller than an average cell size within a specimen are formed at least one of intracellular or extracellular particulate sites in the specimen.

17. The cryoencapsulating method of claim 11 wherein toxic cryoprotectant damage to a specimen is reduced or avoided by a cryoprotectant being captured within any one or more of crystalline ice, crystalline-ice grain boundaries, vitreous ice or vitreous-ice boundaries.

18. The cryoencapsulating method of claim 11 wherein a frozen specimen has superior mechanical toughness, fracture resistance or stress-relief behavior compared to the same specimen without the particulate.

19. The cryoencapsulating method of claim 11 wherein the method is adapted to the cryoencapsulation of one or more of:
a) human or animal tissues or body fluids;
b) organs or biological tissues;
c) plants or plant derivatives;
d) food or food derivatives;
e) marine life or derivatives thereof;
f) seeds or pollen;
g) a tissue, fluid or material containing genetic information;
h) fruits or derivatives thereof;
i) vegetables or derivatives thereof;
j) body parts, tissues or fluids for intended transplant or reimplant; or
k) cellular matter, whether natural or human engineered.

20. A cryoencapsulated natural or manmade biological specimen structure comprising:
a) a specimen, including a natural or manmade biological or genetic material;
b) a particulate material dispersed in the specimen;
c) at least one cryoprotectant infused into the specimen;
d) the specimen containing the particulate and cryoprotectant maintained at a temperature at or below 0 Deg C.;
e) the dispersed particulates substantially each spatially associated with:
i) a local region of crystalline or vitreous ice formation, or
ii) a particle-targeted specimen portion.

21. The cryoencapsulated specimen structure of claim 20 wherein the dispersed particulate is for at least one of:
a) nucleation of ice crystals;
b) encouragement of at least some vitreous-ice formation rather than crystalline-ice formation;
c) targeting to specific specimen portions for their protection;
d) use as a solid, semisolid, liquid or gaseous particulate; or
e) use as a targeted cryoprotectant molecule.

22. The cryoencapsulated specimen structure of claim 20 wherein at least one of:
a) ice crystals have a size substantially smaller than an average biological cell size in the specimen;
b) ice crystals or ice crystal grain boundaries as a group have a large specific surface area which incorporates cryoprotectant;
c) the formation of vitreous ice is encouraged over crystalline ice because the particulate interferes with ice crystallization or water mobility; or
d) a particle includes a cryoprotecting material which is targeted to a specific specimen portion for its specific cryoprotection.

23. The cryoencapsulated specimen of claim 20 wherein at least some extracellular space in a biological specimen has a cold-ductile cryoprotecting material infused therein thereby imparting global toughness, stress relief or fracture resistance to the cryoencapsulated specimen.

24. The cryoencapsulated specimen of claim 20 wherein the particles are any of:
a) solid, semisolid or liquid microparticles or nanoparticles;
b) human-engineered or manmade, biological, inorganic or organic microparticles or nanoparticles;
c) particles which are precipitated or formed in the specimen;
d) particles which are infused or otherwise delivered into the specimen;
e) gas microbubbles or nanobubbles whether nucleated from a supersaturated liquid or nucleated via acoustic cavitation; or
f) targeted cryoprotecting species or molecules.

25. A targeted cryoprotectant for use in preserving natural or manmade biological specimens, materials, tissues or genetic-entity-containing materials comprising:
a) a cryoprotecting species introducible into said specimen;
b) the cryoprotecting species having or also incorporating a molecular feature which serves to attach or bind the cryoprotectant to a specific type of target site in said specimen; and
c) at least some targeted sites of said specimen being cryoprotected by the bound targeted cryoprotectant.
